(19) United States
(12) Patent Application Publication
(10) Pub. No.: US 2022/0330888 A1
(43) Pub. Date: Oct. 20, 2022

(54) ACCESSORIES FOR A MANDIBLE ADVANCEMENT DEVICE

(71) Applicant: Prosomnus Sleep Technologies Inc., Pleasanton, CA (US)

(72) Inventors: Sung KIM, Pleasanton, CA (US); David W. KUHNS, Pleasanton, CA (US); Leonard A. LIPTAK, Pleasanton, CA (US)

(21) Appl. No.: 17/640,775

(22) PCT Filed: Dec. 11, 2020
(86) PCT No.: PCT/US2020/064678

§ 371 Date: Mar. 4, 2022

Related U.S. Application Data
(60) Provisional application No. 62/947,398, filed on Dec. 12, 2019.

(51) Int. Cl. A61B 5/00; A61B 5/11; A61B 5/0205; A61B 5/08

(52) U.S. Cl. CPC A61B 5/682; A61B 5/0022; A61B 5/1114; A61B 5/02055; A61B 5/08; A61B 2560/0214

(57) ABSTRACT

Mandibular advancement devices (MAD) are disclosed comprising an upper splint, a lower splint, and at least one sensor, wherein the sensor measures a biological or biophysical aspect of a patient. Methods of using the devices are also disclosed.

### RELATED APPLICATIONS

The present application claims priority to the U.S. Provisional Application Ser. No. 62/947,398, filed Dec. 12, 2019, by KIM et al., and entitled “ACCESSORIES FOR A MANDIBLE ADVANCEMENT DEVICE,” the entire disclosure of which, including any drawings, is incorporated by reference herein.

### FIELD OF THE INVENTION

The present invention is in the field of dental devices. In particular, the present invention is in the field of a computer aided design procedure for preparing a design and manufacturing a dental device.

### BACKGROUND OF THE DISCLOSURE

Mandibular advancement devices (MAD) have a long residence time inside a patient's mouth, namely throughout the time the patient is asleep. During this time, many physiological and physical changes occur in the patient's body that affect the efficacy of the MAD and the patient's health. It is useful to allow the MAD respond in real time to the changes in the patient in order to provide the most effective mandibular position adjustment for the patient at the particular time. In addition, physicians would like to know the history of the changes in the patient's body while the MAD is being used in order to provide a better treatment regimen.

### SUMMARY OF THE INVENTION

Mandibular advancement devices (MAD) are disclosed comprising an upper splint, a lower splint, and at least one sensor, wherein the sensor measures a biological or biophysical aspect of a patient. Methods of using the devices are also disclosed.

### DETAILED DESCRIPTION OF THE EMBODIMENTS

Disclosed herein are sensors that, when combined with a mandibular advancement device (MAD), can measure certain biological or biophysical aspects about a patient.

One MAD contemplated to be used in combination with the presently disclosed sensors has been previously described. See, for example, U.S. Pat. Nos. 9,820,882, 9,808,327, US Patent Application Publications, 2018/0024530 and 2019/0105191, International Publication WO 2019/018309 A1, and International Patent Application No. PCT/US2019/029471. The disclosure of all the publications enumerated in this paragraph (“the above-incorporated publications”) are hereby incorporated by reference herein, including any drawings, in particular such aspects of the disclosure that disclose an MAD, a method of design thereof, a method of manufacture thereof, or an accessory therefor.

Advantages of the MAD-sensor combinations disclosed herein are many. The device in its totality is 100% self-contained and resides completely secure inside the patient's mouth, enabling complete lip seal. The device is custom manufactured—by combining the patient's anatomical data input and a health care provider's (HCP) prescription with a manufacturing library of elements—to seamlessly integrate the sensors and device mechanisms with the patient's comfort in mind.

In some embodiments, the HCP's prescription includes the starting bite position, and optionally one or more of custom treatment features, such as anterior discluder, splint options, titration mechanism, and other features enumerated in the above-incorporated publications.

In some embodiments, if the MAD comprises more than one sensor, then the MAD is designed and manufactured such that the sensors are on one splint, either the upper or the lower splint, of the MAD. In other embodiments, the sensors are on different splints. Throughout the present disclosure, the splint bearing the sensor(s) is called the “technical splint,” while the splint without any sensors is called the “free splint.”

In some embodiments, the splints are designed such that the most critical components, e.g., the sensors, are protected. If there is to be a device failure due to, for example, the patient bruxing, chewing, or biting against the device, the free splint is designed to fail first before the technical splint. This is achieved by either making the free splint thinner than the technical splint, or by making the two splints from different materials. The free splint is easily remade at a lower cost and more expediently than the technical splint.

### Sensors

A variety of sensors are contemplated to be used with the disclosed MADs. In general, the sensors can be divided into the following categories: physiological sensors, physical sensors, chemical sensors, and positional sensors.

In some embodiments, physiological sensors measure and relay such physiological data as body temperature, respiration rate, heart rate, or other relevant physiological data, and/or any variability in the above values.

In some embodiments, physical sensors are those that detect the mode of breathing, e.g., vibration in the breathing, airflow rate, oxygen concentration of inhaled air, etc., and correlate that to airflow restriction. In some embodiments, the sensor is selected from an oxygen sensor measuring the oxygen concentration of the inhaled air, a carbon dioxide sensor measuring the carbon dioxide concentration of the exhaled air, a pressure sensor measuring the atmospheric pressure or the air pressure inside the oral cavity, an airflow sensor, a noise detector, or an actigraphy sensor. The sensors can also detect snoring, and/or perform airway flow signature analysis. In some embodiments, the sensor measures the pressure exerted on the MAD by the patient's teeth in order to measure the extent of clenching and/or grinding of dentition surfaces.

In some embodiments, chemical sensors are used to measure the body's physiological response to breathing. For example, the saliva pH, saliva sugar concentration, saliva conductivity, levels of stress markers, such as salivary cortisol, blood oxygen saturation level, blood pH, blood glucose levels, blood insulin levels, inflammatory markers, and the like, can be measured in real time and reported to the HCP via the base. Bacterial biosensors can provide information to the HCP on the level of bacterial activity in the mouth during sleep.

In some embodiments, positional sensors are used to record the position of the MAD in the mouth with respect to a predetermined reference location. The sensors can optionally track the movement of the mandible in the anterior-posterior, vertical, and/or lateral directions.

In some embodiments, the sensor is a component of a sensing block, which includes other components besides the sensor. In certain embodiments, the sensing block components include one or more of a battery (rechargeable or replaceable), a battery recharging circuit compatible with industry standards, if applicable, on-board memory, communication module, analog/digital converter to convert sensor voltage inputs to digital signal, a control module for activating stepper motors, and an I/O bus to connect to external sensors and motors.

In some embodiments, during the design of the MAD having at least one sensor, the sensor and the pocket into which it is placed are design library elements in computer aided design (CAD) program. The designer chooses the relevant library element and the location where the sensor should be placed on the MAD and the CAD program provides a design accordingly. For a discussion of designing MADs using library elements, see the above-incorporated publication 2018/0024530.

### Communication

The presently disclosed sensors are in wireless communication with a base, transmitting the data they obtained. Various modes of wireless communication are well-known in the art. Currently, the most popular mode appears to be Bluetooth® communication. Other modes such as radio, infrared, magnetic, or the like can also be used. All modes of wireless communication now known or developed in the future are contemplated for use with the presently disclosed sensors.

In some embodiments, the base is a software contained in a physical cradle. The cradle is configured for wireless communication with the sensor embedded in the MAD. In some embodiments, following the use, the patient places the MAD in the cradle, which can optionally recharge the batteries of the sensor. In some embodiments, the cradle is configured to clean the MAD, for example, by providing a bath into which the MAD can be placed, or by having a well-contained chamber for the MAD to be cleaned using cleansers or steam or the like. In other embodiments, the base is a software (including an app) on a smart phone (e.g., iPhone®, Galaxy®), smart tablet (e.g., iPad®, Surface®), or a laptop or desktop computer (collectively “a device”).

In some of the embodiments, the base logs the position of the mandible in time. The HCP can then correlate the physiological response at a certain time point with the mandibular position at that time point and make treatment decisions accordingly.

In some embodiments, the base and the MAD operate in a feedback system. When the data obtained by the sensor is communicated with the base, the base analyzes the data and, based on a pre-scripted routine, relays a command to the MAD to take an action in response. In some embodiments, the pre-scripted routine is based on a prescription by the HCP.

In some embodiments, the response is a mechanical response. In these embodiments, the MAD comprises a stepper motor, for example embedded as dorsal style, which can cause the mandible to advance or retract either symmetrically or asymmetrically, by 0.1 mm, or any other multiple thereof.

In some embodiments, the base will sound an alarm audible enough to wake the patient up, if the physiological data, such as the blood oxygen level or air flow disturbance, indicate an unhealthy state for the patient to continue to be sleeping. In other embodiments, the base relays a command to the MAD to release a repugnant chemical substance in the mouth, such as one with a bitter taste, to wake the patient up.

In some embodiments, the base is programmed to alert the emergency medical services if the physiological data is worsening and the patient shows no sign of waking up, for example, by turning the alarm off.

In some embodiments, the presently disclosed combination of MAD and sensors is used to deliver medications to the patient in a controlled fashion. In some of these embodiments, the MAD comprises built-in refillable cavities that can be filled with a prescribed medication. In other embodiments, the MAD comprises a location for a prefilled container of medication to be placed. In any case, the design of the MAD with the medication-dispensing components is such that the patient does not feel the bulk of the medication-dispensing components and the MAD is as comfortable to wear as if it did not have the medication-dispensing components.

In response to a time cue or input from an embedded sensor, the base relays a command to the MAD and the medication-dispensing components to release a preset amount of the medication either between the cheek and the gum for a buccal administration or into the patient's mouth for the medication to be inhaled. Examples include stress reducing agents, calming agents, glucose, insulin, nitroglycerin or other heart medications for atrial fibrillation or unstable angina, and the like.

In some embodiments, such as those where the MAD is as described in the above-incorporated International Patent Application No. PCT/US2019/029471, the MAD comprises an internal mechanism to advance the mandible forward, for example by turning a screw. In some of these embodiments, a small stepper motor is connected with the advancement mechanism. In some embodiments, in response to data obtained from the sensors, for example with respect to snoring, whether the flow of air through the mouth is laminar or turbulent, heart rate variability, blood oxygen saturation levels, and the like, the base sends a command to the MAD stepper motors to advance or retract the mandible by a small increment until the situation is rectified, for example the air flow becomes laminar, or snoring subsides, or blood oxygen levels rise. The biofeedback provided by the sensors allow the base to control the treatment in real time.

In some embodiments, the base is also in wireless communication with a software on a device operated by a health care provider (HCP). In these embodiments, the base communicates the collected data directly to the HCP device, where the HCP can monitor the progress of the patient without the need for the patient to make office visits. This feature is quite useful for individuals who travel constantly, such as salespersons, long distance drivers, airline pilots, and the like. By taking advantage of this feature, the HCP can continually monitor the patient and intervene with a recommendation if that is in the best interest of the patient. This way, problems are detected and corrected as they happen.

In some embodiments, the base communicates with the HCP software through the internet, phone lines, satellite, radio, microwave, or other forms of long distance communication now known or later developed.

In some embodiments, the base can analyze the data and, in accordance with a pre-scripted routine, cause the MAD to change the position of the mandible with respect to maxilla to maximize the efficacious result during the use of the MAD.

In some embodiments, multiple sensors are connected to the same sensing block, whereas in other embodiments, each sensor has its own sensing block.

To adjust the MAD to advance the mandible when such advancement is required, MADs currently on the market require the patient to turn a screw a number of times for the proper adjustment. When the screw is turned, a mobile unit of the MAD moves with respect to a stationary unit thereof. In many instances, each adjustment requires the screw to be turned more than twice. Patient compliance with this directive is not always 100% as some patients become distracted and forget how many times they turned the screw, or the screw is not turned all the way, and other similar problems.

In some embodiments, the MAD comprises barcodes that can be read by a barcode reader, such as a smart phone. QR codes, standard barcodes, and other similarly readable figures can be used for the present purpose. A portion of the barcode is printed on the mobile unit and the rest of the barcode is printed on the stationary unit. At each unit increments, such as 0.1 mm, the two halves of the barcode align to create a code that corresponds to the position of the mobile unit with respect to the stationary unit. When the device has not been properly adjusted, the resulting barcode is a garble and does not result in any information being received. When the device is properly adjusted, the alignment of two halves of the barcode correspond to the code for the position of the device.

1: A mandibular advancement device (MAD) comprising an upper splint fabricated to fit onto an upper dentition of an individual in need thereof, a lower splint fabricated to fit onto a lower dentition of the individual in need thereof, and at least one sensor, wherein the sensor measures a biological or biophysical aspect of a patient, and the at least one sensor is attached to or incorporated into or onto the upper splint or the lower splint.

2: The mandibular advancement device of claim 1, wherein the at least one sensor measures a biological or biophysical aspect requested by a health care professional in a prescription.

3: The mandibular advancement device of claim 2, wherein the prescription comprises modifying a starting bite position.

4: The mandibular advancement device of claim 3, wherein the custom treatment feature comprises an anterior discluder, splint options, or a titration mechanism.

5: The mandibular advancement device of claim 1, wherein the device comprises two or more sensors, and wherein all the sensors are on one splint, either the upper or the lower splint, of the MAD.

6: The mandibular advancement device of claim 1, wherein the device comprises at least two or more sensors, and wherein one or the at least two or more sensors is attached to or incorporated into or onto the upper splint and one of the at least two or more sensors is attached to or incorporated into or onto the lower splint.

7: The mandibular advancement device of claim 1, wherein the at least one sensor comprises a physiological sensor, a physical sensor, a chemical sensor, or a positional sensor.

8: The mandibular advancement device of claim 7, wherein the physiological sensor measures patient data comprising blood oxygen levels, body temperature, respiration rate, or heart rate.

9: The mandibular advancement device of claim 7, wherein the physical sensor detects vibration in the breathing, airflow rate, oxygen concentration of inhaled air, carbon dioxide concentration of exhaled air, atmospheric pressure, air pressure inside the patient's oral cavity, noise, pressure exerted on the MAD by the patient's teeth, or actigraphic data.

10: The mandibular advancement device of claim 7, wherein the chemical sensor detects saliva pH, saliva glucose concentration, saliva conductivity, stress markers, salivary cortisol, blood oxygen saturation level, blood pH, blood glucose levels, blood insulin levels, or inflammatory markers.

11: The mandibular advancement device of claim 7, wherein the positional sensor detects and records a position of the MAD in a mouth with respect to a predetermined reference location.

12: The mandibular advancement device of claim 1, wherein the at least one sensor is a component of a sensing block, and the sensing block comprises at least one additional component, the additional component comprising a rechargeable or replaceable battery, a battery recharging circuit compatible with industry standards, an on-board memory, a communication module, an analog/digital converter, a control module for activating stepper motors, or an I/O bus to connect to external components.

13: The mandibular advancement device of claim 1, wherein the at least one sensor comprises a communication component configured for wireless communication with a base.

14: The mandibular advancement device of claim 13, wherein the wireless communication is in the form of a radio, an infrared, or a magnetic communication.

15: The mandibular advancement device of claim 13, wherein the base comprises software, optionally software on an app, a smartphone, a smart tablet, a laptop computer or a desktop computer.

16: The mandibular advancement device of claim 15, wherein the base software logs the position of the lower dentition in time.

17: The mandibular advancement device of claim 13, wherein the base and the mandibular advancement device operate in a feedback system, and when data obtained by the at least one sensor is communicated with the base, the base analyzes the data and, based on a pre-scripted routine, relays a command to the mandibular advancement device to take an action in response.

18: The mandibular advancement device of claim 17, wherein the mandibular advancement device further comprises a motor, and the action comprises a communication to the motor from the base to activate a change in the positional relationship of the upper splint positioned on the upper dentition to the lower splint positioned on the lower dentition.

19: A method for repositioning a mandible, comprising placing a mandibular advancement device of claim 1, onto an upper and a lower dentition of an individual in need thereof.

20: The method of claim 19, wherein the mandibular advancement device further comprises a motor,
wherein the at least one sensor comprises a communication component configured for wireless communication with a base,
and the base and the mandibular advancement device operate in a feedback system, and when data obtained by the at least one sensor is communicated with the base, the base analyzes the data and, based on a pre-scripted routine, relays a command to the mandibular advancement device to take an action in response,
and the action comprises a communication to the motor from the base to activate a change in the positional relationship of the upper splint positioned on the upper dentition to the lower splint positioned on the lower dentition.
